(19) United States
(12) Patent Application Publication
(10) Pub. No.: US 2024/0423502 A1
(43) Pub. Date: Dec. 26, 2024

(54) GAIT TRAINING SYSTEM, CONTROL METHOD, AND NON-TRANSITORY STORAGE MEDIUM

(71) Applicant: Toyota Jidosha Kabushiki Kaisha, Toyota-shi (JP)

(72) Inventor: Taiga MATSUMOTO, Nagoya-shi (JP)

(73) Assignee: Toyota Jidosha Kabushiki Kaisha, Toyota-shi (JP)

(21) Appl. No.: 18/662,268

(22) Filed: May 13, 2024

(30) Foreign Application Priority Data
Jun. 26, 2023 (JP) 2023-103971

(51) Int. Cl. A61B 5/11; A61B 5/103; A63B 22/02

(52) U.S. Cl. CPC A61B 5/112; A61B 5/1036; A63B 22/02

(57) ABSTRACT

A gait training system according to an embodiment includes: a treadmill having a belt on which a user lands; a load distribution sensor that has a plurality of sensors arranged in a two-dimensional array and detects a load applied through the belt; a sensor output acquisition unit that acquires sensor outputs from the sensors of the load distribution sensor; a correction unit that corrects the sensor outputs from the respective sensors based on a moving speed of the belt; and a value-of-load calculation unit that calculates a value of a load received from the user based on corrected values corrected by the correction unit.

### CROSS-REFERENCE TO RELATED APPLICATION

This application claims priority to Japanese Patent Application No. 2023-103971 filed on Jun. 26, 2023, incorporated herein by reference in its entirety.

### BACKGROUND

### 1. Technical Field

The present disclosure relates to a gait training system, a control method, and a non-transitory storage medium.

### 2. Description of Related Art

Japanese Unexamined Patent Application Publication No. 2007-218892 (JP 2007-218892 A) discloses a pressure distribution detection device. This pressure distribution detection device includes a plurality of driving coils and a plurality of detection coils. It further includes spacer means that keeps the distance between the driving coils and the detection coils constant. The driving coils are provided with a cushion material and an electromagnetic shielding body. The detection coils are provided with a cushion material and a variable electromagnetic coupling body. The variable electromagnetic coupling body is a magnetic sheet having a sheet shape.

### SUMMARY

When detecting a pressure distribution, a pressure (a load) is detected based on an amount of deformation (deforming stress) of a viscoelastic body. When the viscoelastic body is formed in a sheet shape, the viscoelastic body deforms also at a region surrounding a site to which a load has been actually applied. As the load is detected also at the surrounding region to which the load has not been applied, it is difficult to detect the load with high accuracy.

In a gait training device using a treadmill or the like, a timing in a walking cycle is inferred according to a load distribution. Driving control is performed according to the timings in the walking cycle. Therefore, by detecting the load distribution with high accuracy, more appropriate driving control can be performed.

Having been devised in view of these circumstances, the present disclosure aims to provide a gait training system, a control method, and a non-transitory storage medium that can appropriately calculate a value of a load.

A gait training system according to an embodiment includes: a treadmill having a belt on which a user lands; a load distribution sensor that has a plurality of sensors arranged in a two-dimensional array and detects a load applied through the belt; a sensor output acquisition unit that acquires sensor outputs from the sensors of the load distribution sensor; a correction unit that corrects the sensor outputs from the respective sensors based on a moving speed of the belt; and a value-of-load calculation unit that calculates a value of a load received from the user based on corrected values corrected by the correction unit.

In the above-described gait training system, the correction unit may determine a sensor at a front end of a region on which the user has landed on a line-by-line basis, and correct the sensor output according to a sensor address in a front-rear direction with reference to a sensor address of the sensor at the front end.

In the above-described gait training system, the correction unit may correct the sensor outputs of the sensors in one row along the front-rear direction using a correction factor according to the sensor address in the front-rear direction.

In the above-described gait training system, the load distribution sensor may sequentially read outputs of the sensors in one row arranged in the front-rear direction.

The above-described gait training system may further include a robot leg worn on a leg of the user, and a driving control unit that drives the robot leg based on the value of the load.

In the above-described gait training system, the value-of-load calculation unit may have a CNN that performs a convolution process using two-dimensional data including the corrected values of the plurality of cells as an input.

A control method of a gait training system according to an embodiment is a control method of a gait training system including a treadmill having a belt on which a user lands, and a load distribution sensor that has a plurality of sensors arranged in a two-dimensional array and detects a load applied through the belt. The control method includes the steps of: acquiring sensor outputs from the sensors of the load distribution sensor; correcting the sensor outputs from the respective sensors based on a moving speed of the belt to obtain corrected values; and calculating a value of a load received from the user based on the corrected values.

In the above-described control method of a gait training system, in the step of correcting, a sensor at a front end of a region on which the user has landed may be determined on a line-by-line basis, and the sensor output may be corrected according to a sensor address in a front-rear direction with reference to a sensor address of the sensor at the front end.

In the above-described control method of a gait training system, the sensor outputs of the sensors in one row along the front-rear direction may be corrected using a correction factor according to the sensor address in the front-rear direction.

In the above-described control method of a gait training system, the load distribution sensor may sequentially read outputs of the sensors in one row arranged in the front-rear direction.

In the above-described control method of a gait training system, the gait training system may further include a robot leg worn on a leg of the user, and a driving control unit that drives the robot leg based on the value of the load.

In the above-described control method of a gait training system, in the step of calculating the value of the load, a CNN may perform a convolution process using two-dimensional data including the corrected values of the plurality of cells as an input.

A non-transitory storage medium according to an embodiment is a non-transitory storage medium storing a control program that makes a computer execute a control method for controlling a gait training system including a treadmill having a belt on which a user lands, and a load distribution sensor that has a plurality of sensors arranged in a two-dimensional array and detects a load applied through the belt. The control method includes the steps of: acquiring sensor outputs from sensors of the load distribution sensor; correcting the sensor outputs from the respective sensors based on a moving speed of the belt to obtain corrected values; and calculating a value of a load received from the user based on the corrected values.

According to this disclosure, it is possible to provide a gait training system, a control method, and a non-transitory storage medium that can appropriately calculate a value of a load.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic perspective view of a gait training system;

FIG. 2 is a sectional side view showing the configuration of a treadmill;

FIG. 3 is a top view schematically showing the configuration of a load distribution sensor and a belt;

FIG. 4 is a sectional view schematically showing deformation of a viscoelastic sheet in the load distribution sensor;

FIG. 5 is sectional views schematically showing deformation of the viscoelastic sheet during operation of the treadmill;

FIG. 6 is a functional block diagram showing main components of a control system of a system;

FIG. 7 is a view for describing arrangement of cells and leading cells in respective lines;

FIG. 8 is graphs showing sensor outputs before correction, and corrected values obtained by a correction unit; and

FIG. 9 is a view for describing a learning model used in a value-of-load calculation unit.

### DETAILED DESCRIPTION OF EMBODIMENTS

The present disclosure will be described below through an embodiment of the disclosure, but this is not to limit the disclosure according to the claims to the following embodiment. Not all of configurations described in the embodiment are necessarily essential as means for solving the problem. To clarify the description, the following text and the drawings are partly omitted and simplified as appropriate. The same elements in the drawings are denoted by the same reference sign, and overlapping description is omitted as necessary.

### System Configuration

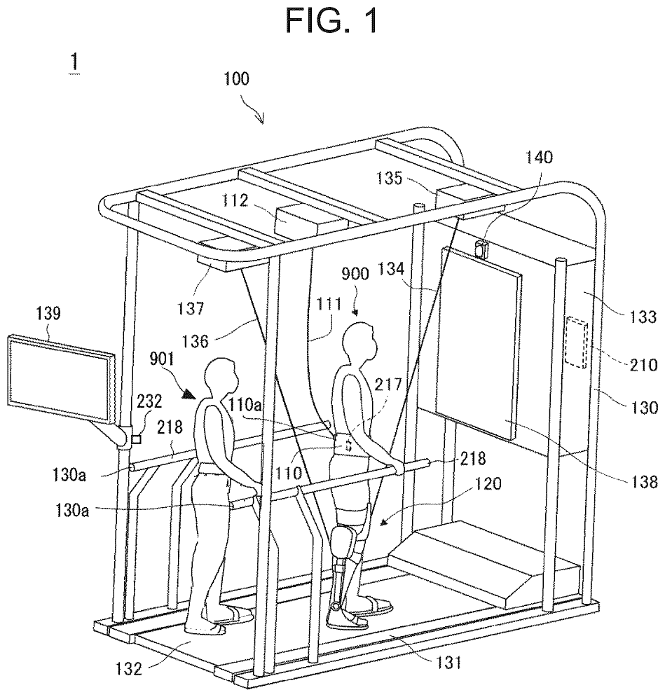

FIG. 1 is an overall conceptual view showing one example of the configuration of a rehabilitation support system according to the embodiment. The rehabilitation support system (gait training system 1) according to this embodiment is composed mainly of a gait training device 100 and a leg brace 120.

The gait training device 100 is one specific example of rehabilitation support devices that support rehabilitation of a trainee (user) 900. The gait training device 100 is a device for the trainee 900 who is a hemiplegic patient suffering paralysis in one leg to undergo gait training in accordance with instructions of training staff 901. Here, the training staff 901 can be a therapist (a physical therapist) or a doctor, and helps the trainee in training through instructions, assistance etc.; therefore, the training staff 901 can also be called a training instructor, a training assistant, a training helper, etc.

The gait training device 100 mainly includes a control panel 133 mounted on a frame 130 that forms an overall framework, and a treadmill 131 on which the trainee 900 walks. Further, the leg brace 120 is worn on the affected leg of the trainee 900 that is the leg on the paralyzed side. In FIG. 1, the leg brace 120 is worn on the right leg of the trainee 900. The leg brace 120 is a robot leg including an actuator etc. For example, the leg brace 120 has a joint driving mechanism that assists the trainee 900 in moving the knee joint.

The frame 130 is erected on the treadmill 131 that is installed on a floor surface. The treadmill 131 rotates a ring-shaped belt 132 by a motor (not shown). The treadmill 131 is a device that prompts the trainee 900 to walk, and the trainee 900 who undergoes walking training rides on the belt 132 and tries to perform a walking motion according to the movement of the belt 132. While the training staff 901 can also perform a walking motion together by, for example, standing on the belt 132 behind the trainee 900 as 25 shown in FIG. 1, it is normally preferable that the training staff 901 be in such a state that he or she can easily assist the trainee 900 by, for example, standing in a state of straddling the belt 132. As will be described later, the treadmill 131 has a load distribution sensor.

The frame 130 supports the control panel 133 and a training monitor 138. The control panel 133 houses an overall control unit 210 that performs control of motors and sensors. The training monitor 138 is, for example, a liquid crystal panel and presents a status of progress of training etc. to the trainee 900. Further, the frame 130 supports a front-side pulling unit 135 near a front side of an overhead part of the trainee 900, a harness pulling unit 112 near the overhead part, and a rear-side pulling unit 137 near a rear side of the overhead part. The frame 130 includes handrails 130a for the trainee 900 to hold.

The handrails 130a are disposed on left and right sides of the trainee 900. Each handrail 130a is disposed in a direction parallel to a walking direction of the trainee 900. Up-down positions and left-right positions of the handrails 130a are adjustable. That is, the handrails 130a can include a mechanism for changing their level and width. Further, the handrails 130a can also be configured such that their inclination angle can be changed by, for example, adjusting their level to a different level between a front side and a rear side in the walking direction. For example, the handrails 130a can be set to such an inclination angle that their level rises gradually along the walking direction.

The handrails 130a are each provided with a handrail sensor 218 that detects a load received from the trainee 900. For example, the handrail sensor 218 can be a load detection sheet of a resistance variation detection type in which electrodes are disposed in a matrix form. Alternatively, the handrail sensor 218 can be a six-axis sensor combining a three-axis acceleration sensor (x, y, z) and a three-axis gyroscope sensor (roll, pitch, yaw). However, the type and the position of installation of the handrail sensor 218 do not matter.

A camera 140 serves a function as an imaging unit for observing the whole body of the trainee 900. The camera 140 is installed near the training monitor 138 so as to face the trainee. The camera 140 takes still images and moving images of the trainee 900 undergoing training. The camera 140 includes a set of a lens and an imaging element with such an angle of view that the whole body of the trainee 900 can be captured. The imaging element is, for example, a complementary-metal-oxide-semiconductor (CMOS) image sensor, and converts an optical image formed in an image formation plane into an image signal.

Through coordinated operation of the front-side pulling unit 135 and the rear-side pulling unit 137, the load of the leg brace 120 is offset such that this load does not put a burden on the affected leg, and further a motion of swinging the affected leg forward is assisted according to a degree of settings.

A front-side wire 134 is coupled at one end to a winding mechanism of the front-side pulling unit 135 and at the other end to the leg brace 120. As a motor (not shown) is turned on and off, the winding mechanism of the front-side pulling unit 135 winds and unwinds the front-side wire 134 according to the movement of the affected leg. Similarly, the rear-side wire 136 is coupled at one end to a winding mechanism of the rear-side pulling unit 137 and at the other end to the leg brace 120. As a motor (not shown) is turned on and off, the winding mechanism of the rear-side pulling unit 137 winds and unwinds the rear-side wire 136 according to the movement of the affected leg. Through such coordinated operation of the front-side pulling unit 135 and the rear-side pulling unit 137, the load of the leg brace 120 is offset such that this load does not put a burden on the affected leg, and further the motion of swinging the affected leg forward is assisted according to the degree of settings.

The front-side wire 134 and the front-side pulling unit 135 constitute first pulling means that pulls the leg of the trainee 900 toward the upper side as well as the front side. The rear-side wire 136 and the rear-side pulling unit 137 constitute second pulling means that pulls the leg of the trainee 900 toward the upper side as well as the rear side. The front-side pulling unit 135 and the rear-side pulling unit 137 pulls the front-side wire 134 and the rear-side wire 136 with a pulling force according to a walking phase as will be described later. An operation pattern for the pulling force may be set according to the walking phase.

For example, as an operator, the training staff 901 sets the level of assistance high for a trainee suffering severe paralysis. When the level of assistance is set high, the front-side pulling unit 135 winds the front-side wire 134 with a comparatively high force according to a timing of swinging forward of the affected leg. When training has progressed and assistance becomes unnecessary, the training staff 901 sets the level of assistance to a minimum level. When the level of assistance is set to the minimum level, the front-side pulling unit 135 winds the front-side wire 134 with a force enough to cancel the weight of the leg brace 120 itself according to the timing of swinging forward of the affected leg.

The gait training device 100 includes a fall-prevention harness device as a safety device that includes a brace 110, a harness wire 111, and the harness pulling unit 112 as main constituent elements. The brace 110 is a belt wrapped around the abdomen of the trainee 900, and is fixed to the hips by means of, for example, a touch-and-close fastener. The brace 110 includes a coupling hook 110a to which one end of the harness wire 111 that is a hanging tool is coupled, and can also be called a hanger belt. The trainee 900 wears the brace 110 such that the coupling hook 110a is located at a back part.

The harness wire 111 is coupled at one end to the coupling hook 110a of the brace 110 and at the other end to a winding mechanism of the harness pulling unit 112. As a motor (not shown) is turned on and off, the winding mechanism of the harness pulling unit 112 winds and unwinds the harness wire 111. When the trainee 900 is about to fall, the fall-prevention harness device thus configured winds the harness wire 111 in accordance with a command from the overall control unit 210 that has detected the movement of the trainee 900, and supports the upper body of the trainee 900 by the brace 110 so as to prevent the trainee 900 from falling.

The brace 110 includes an attitude sensor 217 for detecting an attitude of the trainee 900. The attitude sensor 217 is, for example, a combination of a gyroscope sensor and an acceleration sensor, and outputs an inclination angle of the abdomen on which the brace 110 is worn relative to the direction of gravitational force.

The management monitor 139 is a display and input device that is mounted on the frame 130 and that mainly the training staff 901 monitors and operates. The management monitor 139 is a liquid crystal panel, for example, and has a touch panel provided on its surface. The management monitor 139 displays various menu items relating to settings of training, various parameter values during training, training results, etc. Near the management monitor 139, an emergency stop button 232 is provided. When the training staff 901 presses the emergency stop button 232, the gait training device 100 comes to an emergency stop.

The overall control unit 210 generates rehabilitation data that can include setting parameters relating to the settings of training, various pieces of data relating to movement of the leg that has been output from the leg brace 120 as a training result, etc. This rehabilitation data can include data showing the training staff 901 or his or her years of experience, level of skills, etc., data showing the symptoms, the walking ability, the degree of recovery, etc. of the trainee 900, various pieces of data output from sensors etc. that are provided outside the leg brace 120, etc.

At least part of the control of the overall control unit 210 may be executed by computer programs. For example, the overall control unit 210 includes a memory (non-transitory storage medium) that stores programs, a processor that executes the programs, etc.

The overall control unit 210 can control actuators, such as motors, based on a detection result of the load distribution sensor. For example, the overall control unit 210 determines the phase (timing) in a walking cycle based on a change in the load detected by the load distribution sensor. Based on the determination result, the overall control unit 210 generates a control signal for controlling the actuators. Based on the control signal, the joint driving mechanism of the leg brace 120 performs assistive operation for the knee joint. In this way, appropriate assistive operation can be performed, so that effective training becomes possible. The overall control unit 210 may control the front-side pulling unit 135 and the rear-side pulling unit 137 according to the phase in the walking cycle. Control based on the detection result of the load distribution sensor will be described later.

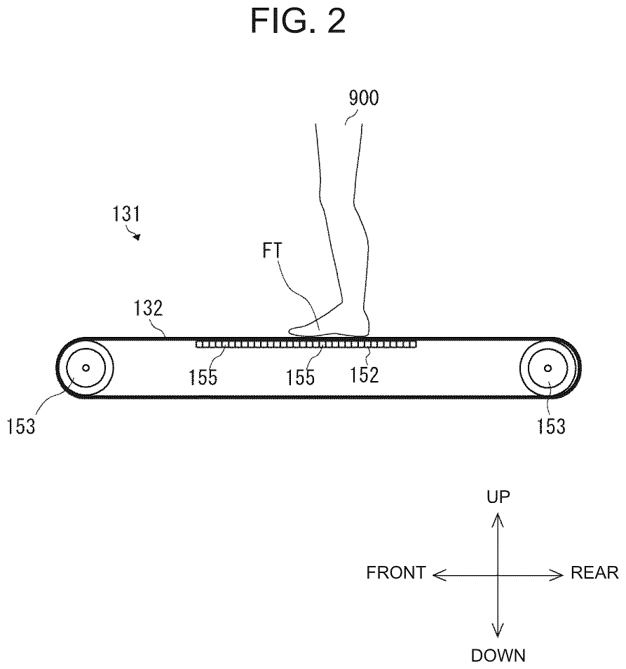

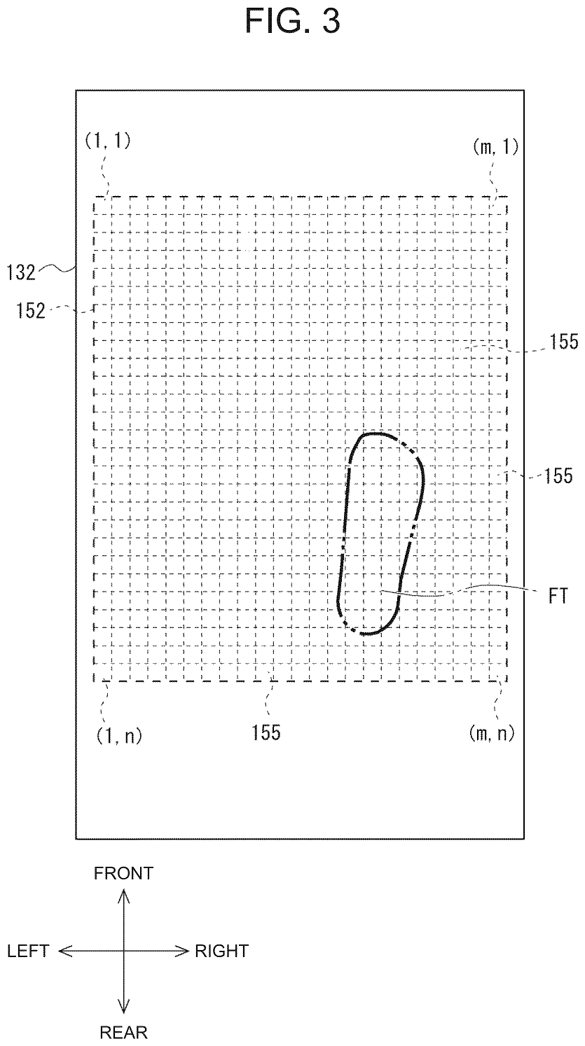

Next, the configuration of the load distribution sensor provided in the treadmill 131 will be described using FIG. 2 and FIG. 3. FIG. 2 is a sectional side view showing the configuration of the treadmill 131 and the load distribution sensor 152. FIG. 3 is a top view schematically showing the arrangement of the load distribution sensor 152 in the treadmill 131.

The treadmill 131 includes at least a ring-shaped belt (moving plate) 132, pulleys 153, and a motor (not shown). The belt 132 is an endless belt and formed in a ring shape as seen in a side view. Inside the ring-shaped belt 132, the two pulleys 153 are disposed. The two pulleys 153 are disposed so as to be spaced apart in a front-rear direction. Rotational axes of the pulleys 153 are parallel to a left-right direction. Thus, when the motor rotates the pulleys 153, the belt 132 is driven in the front-rear direction. When the trainee 900 or the training staff 901 sets the moving speed, the motor rotates the pulleys 153 such that the belt 132 moves at that moving speed.

Further, inside the ring-shaped belt 132 (on the lower side of the surface of the belt 132 on which the trainee 900 rides), the load distribution sensor 152 is disposed so as not to move along with the belt 132. The load distribution sensor 152 is disposed on the inside of the ring of the endless belt. The load distribution sensor 152 has a plurality of sensors 155 as components. The plurality of sensors 155 is disposed in a matrix form on the lower side of the belt 132 that supports the bottom of the foot of the trainee 900 undergoing gait training. By using the plurality of sensors 155, the load distribution sensor 152 can detect a distribution of a contact pressure (a load) received from the foot FT of the trainee 900 riding the belt 132.

As shown in FIG. 3, the load distribution sensor 152 has the plurality of sensors 155 disposed in a matrix form. The load distribution sensor 152 has a sheet shape parallel to a horizontal plane. As the sensors 155, sensors of electromagnetic induction type, electrical resistance type, capacitance type, load cell type, etc. can be used. Of course, the type of the sensors 155 is not particularly limited.

For example, the load distribution sensor 152 has m (m is an integer not smaller than 2) sensors 155 disposed in the left-right direction and n (n is an integer not smaller than 2) sensors 155 disposed in the front-rear direction. In FIG. 3, the XY-coordinates of the sensor 155 at the left front corner are (1, 1), and the XY-coordinates of the sensor 155 at the left rear corner are (1, n). The XY-coordinates of the sensor 155 at the right front corner are (m, l), and the XY-coordinates of the sensor 155 at the right rear corner are (m, n). Each sensor 155 has the same size. Thus, each sensor 155 is assigned an XY-address (XY-coordinates).

The plurality of sensors 155 is disposed at regular intervals in the front-rear direction and the left-right direction. Thus, a sensing region of the load distribution sensor 152 has a rectangular shape. As such, an output from the load distribution sensor 152 is m×n pieces of two-dimensional map data.

One sensor 155 is also called a cell. The load distribution sensor 152 detects a pressure (a load) on a cell-by-cell basis. By sequentially reading outputs of the plurality of sensors 155, the load distribution sensor 152 detects the distribution of the load received from the trainee 900. Each sensor 155 detects a two-dimensional distribution of the pressure received from the foot FT through the belt 132. The m×n pieces of two-dimensional data are also called data of one frame.

Based on the detection result of the load distribution sensor 152, the overall control unit 210 estimates the value of the load from the foot FT. The value of the load here represents the total load received from the entire foot FT. For example, the output values detected by the plurality of sensors 155 can be converted into pressures using a conversion map. The output values can be converted into the value of the load by integrating these pressures. Based on the value of the load, the overall control unit 210 performs determination relating to the walking phase. For example, the overall control unit 210 determines that the walking phase is a timing of transition from a contact leg to an idling leg.

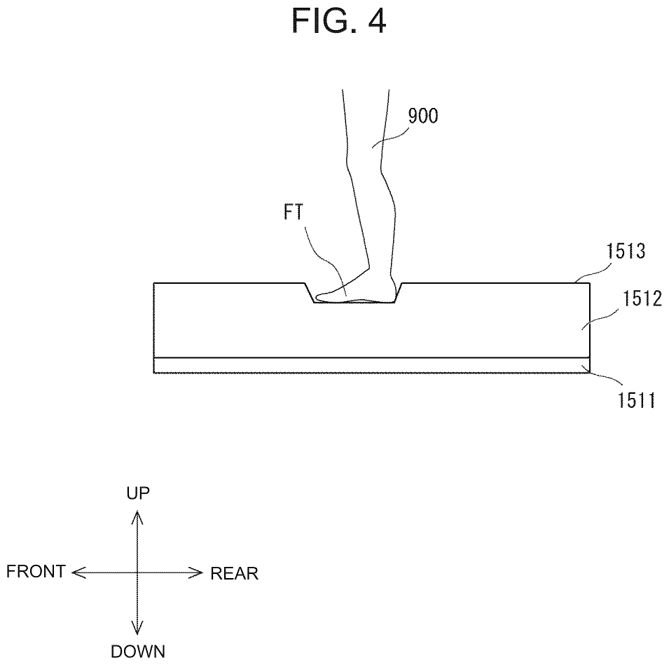

The load distribution sensor 152 is a sheet-shaped sensor and has a viscoelastic body. For example, as shown in FIG. 4, the load distribution sensor 152 has a base plate 1511, a viscoelastic sheet 1512, and a conductive sheet 1513. FIG. 4 is a sectional side view schematically showing the cross-sectional configuration of the load distribution sensor 152. Here, the description assumes that the load distribution sensor 152 is a sensor of electromagnetic induction type. In FIG. 4, the treadmill 131, the belt 132, etc. are not shown. The description assumes that in FIG. 4, the treadmill 131 is not operating.

The load distribution sensor 152 includes the base plate 1511, the viscoelastic sheet 1512, and the conductive sheet 1513. The base plate 1511, the viscoelastic sheet 1512, and the conductive sheet 1513 are disposed in this order from a lower surface side. The viscoelastic sheet 1512 is provided between the conductive sheet 1513 and the base plate 1511 of the load distribution sensor 152. The viscoelastic sheet 1512 is formed by, for example, a resin material, such as urethane foam. The up-down direction is a thickness direction of the viscoelastic sheet 1512.

The base plate 1511 has a coil pattern having a plurality of coils. The coil pattern has coils that are provided for the respective cells. In the base plate 1511, the plurality of coils is arranged in an array. The conductive sheet 1513 is, for example, an aluminum sheet, and is formed over the entire load distribution sensor 152 so as to cover the plurality of coils. The distance between the conductive sheet 1513 and the base plate 1511 changes according to the load. That is, as the load becomes higher, the viscoelastic sheet 1512 is further crushed and becomes thinner. When the viscoelastic sheet 1512 deforms, the electrical characteristics of the base plate 1511 change.

The load distribution sensor 152 can obtain the amount of deformation by measuring the electrical characteristics, such as electromagnetic induction, on a cell-by-cell basis. The load distribution sensor 152 converts the amount of deformation of the viscoelastic sheet 1512 in the thickness direction into a load. The amount of deformation can be converted into a load on a cell-by-cell basis. Thus, as the electrical characteristics change according to the amount of deformation of the viscoelastic sheet 1512, each sensor 155 can detect the load. Of course, the load distribution sensor 152 is not limited to an electromagnetic induction type, and a load distribution sensor of capacitance type, electrical resistance type, etc. may also be used.

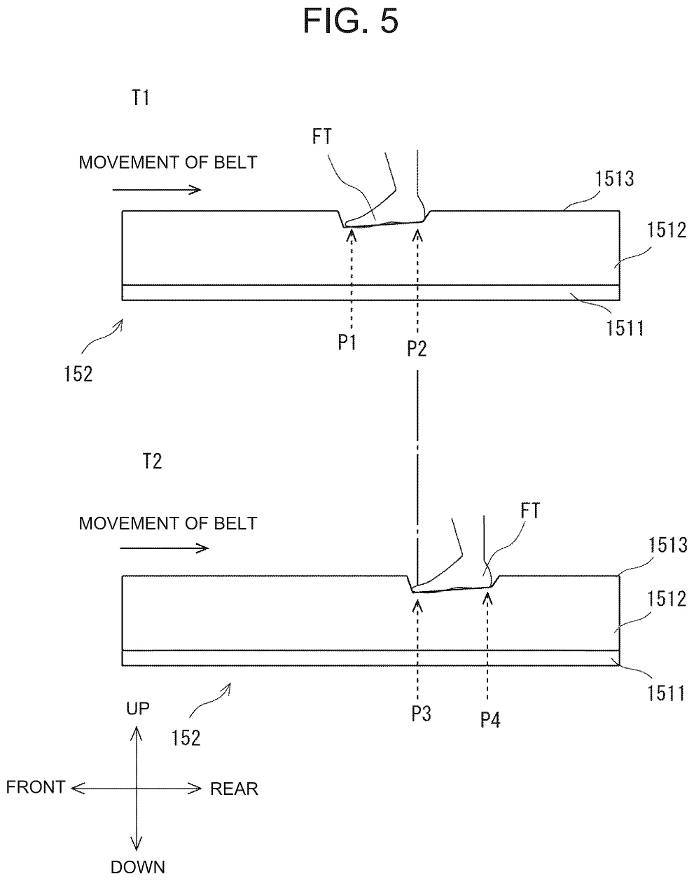

FIG. 5 shows deformation of the viscoelastic sheet 1512 in a state where the treadmill is operating. The left side in FIG. 5 is the front side in the walking direction, and the belt 132 (not shown) is moving from left to right. Since the belt 132 is moving, the detection position of the load distribution sensor moves gradually toward the rear side with the foot FT landed.

For example, at timing T1 when the foot FT has landed, a front-rear position of a front end of the foot FT on the treadmill 131 is a position P1 and a front-rear position of a rear end thereof is a position P2. At timing T2 after a predetermined time has elapsed since the timing T1, the belt 132 has moved with the foot FT landed. Thus, the front-rear position of the front end of the foot becomes a position P3 and the front-rear position of the rear end becomes a position P4.

Here, in the treadmill 131, the position P2 and the position P3 coincide with each other. In this case, at the position P3, a load is constantly applied to the load distribution sensor 152 during the period from the timing T1 to the timing T2. This means that the viscoelastic sheet 1512 is constantly deformed during the period from the timing T1 to the timing T2.

On the other hand, at the position P4, a load is newly applied to the load distribution sensor 152 at the timing T2. Thus, when the timing T2 comes, deformation of the viscoelastic sheet 1512 starts at the position P4. The position P4 is a region at which the viscoelastic sheet 1512 becomes newly crushed. At the position P4, therefore, it takes time before the viscoelastic sheet 1512 becomes completely crushed.

As the belt 132 moves, a load is newly applied at the rear end of the foot FT. On the other hand, a site that has been directly under the front end of the foot FT is located directly under the rear end of the hoot FT as a result of movement of the belt. In such a case, the relationship between the applied load that is actually applied and the detected load that is the sensor output differs between the front end and the rear end of the foot FT. For example, even when a uniform load is received from the entire foot FT, the sensor output of the cell at the front end of the foot may be larger than the sensor output of the cell at the rear end.

For this reason, the value of the load may fail to be detected with good accuracy. When the detection accuracy of the value of the load is low, the timing of driving the leg brace 120 may differ from the timing in the walking cycle. In this embodiment, therefore, a process to be shown below is performed to estimate the value of the load with high accuracy. Specifically, the overall control unit 210 corrects the sensor outputs according to the moving speed of the belt 132. In the following, the process according to this embodiment will be described.

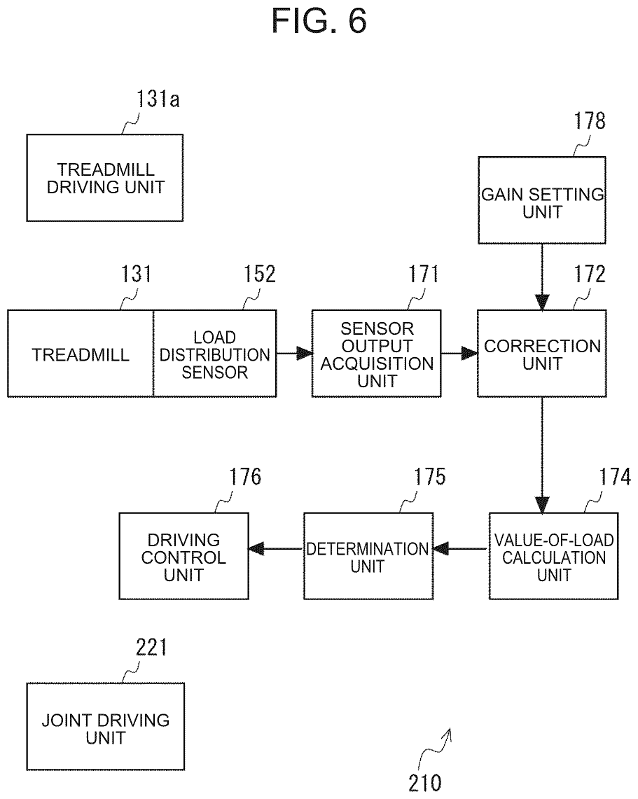

FIG. 6 is a functional block diagram for describing the control according to this embodiment. The overall control unit 210 includes a sensor output acquisition unit 171, a correction unit 172, a value-of-load calculation unit 174, a determination unit 175, and a driving control unit 176. The joint driving unit 221 is provided in the leg brace 120. Specifically, the joint driving unit 221 includes a motor and a rotary mechanism for assisting the trainee 900 in moving the knee joint.

The treadmill driving unit 131a drives the treadmill 131. The treadmill driving unit 131a has a motor etc. for rotating the pulleys 153. The treadmill 131 operates such that the belt 132 moves at a moving speed set by the training staff 901 etc.

The treadmill 131 is provided with the load distribution sensor 152. The load distribution sensor 152 outputs a detection signal (a sensor output) showing a detection result to the overall control unit 210. The sensor output may be digital data or may be analog data. That is, the load distribution sensor 152 may have an A/D converter that converts an analog detection signal into digital data. Or the sensor output acquisition unit 171 may have an A/D converter.

The sensor output includes the output values of the respective cells. The sensor output acquisition unit 171 acquires the sensor output from the load distribution sensor 152 on a cell-by-cell basis. That is, in the data showing the sensor output, the address and the output value of the cell are associated with each other. The sensor output acquisition unit 171 may acquire the data of the sensor outputs in the order of being read by the load distribution sensor 152. The sensor outputs from all the cells constitute data of one frame. The load distribution sensor 152 reads the data line by line or cell by cell.

The correction unit 172 corrects the cell outputs of the load distribution sensor 152 based on the moving speed of the belt 132 of the treadmill 131. The correction unit 172 uses a correction formula based on the cell address of the load distribution sensor 152. The correction unit 172 corrects the sensor outputs using the correction formula.

The value of a sensor output after correction is referred to as a corrected value C. When the cell output before correction is S and a correction factor is a (L, V), the corrected value C is expressed by the following Formula (1):

$$C = S \times α ( L , V ) \qquad ( 1 )$$

L is a cell number in the front-rear direction from a cell serving as a reference (a leading cell 156 to be described later), and V is a treadmill speed. The cell serving as a reference can be the cell at the front end of the region on which the foot FT has landed. The correction factor α is a factor that varies with the cell number L and the treadmill speed V. Specifically, α (L, V) can be as in the following Formula (2):

$$α ( L , V ) = ( 1 + L \times β + V \times γ ) \qquad ( 2 )$$

β and γ are recorded in the gain setting unit 178. β is a gain in a forward direction that is set beforehand, and can be a constant value. γ is a gain of the treadmill speed that is set beforehand, and is a constant value. Each of β and γ is a number larger than 0. Thus, as the moving speed of the belt 132 becomes higher, the correction factor α becomes larger. Further, as the cell number L becomes larger, the correction factor α becomes larger. The correction unit 172 corrects the sensor outputs of each line based on Correction Formula (1). The correction unit 172 can thereby appropriately correct the sensor outputs.

Alternatively, the correction factor α (L, V) for obtaining the corrected value C may be as in the following Formula (3). That is, a shown in Formula (3) can be used instead of a shown in Formula (2):

$$α ( L , V ) = ( 1 + ( V \times γ ) L ) \qquad ( 3 )$$

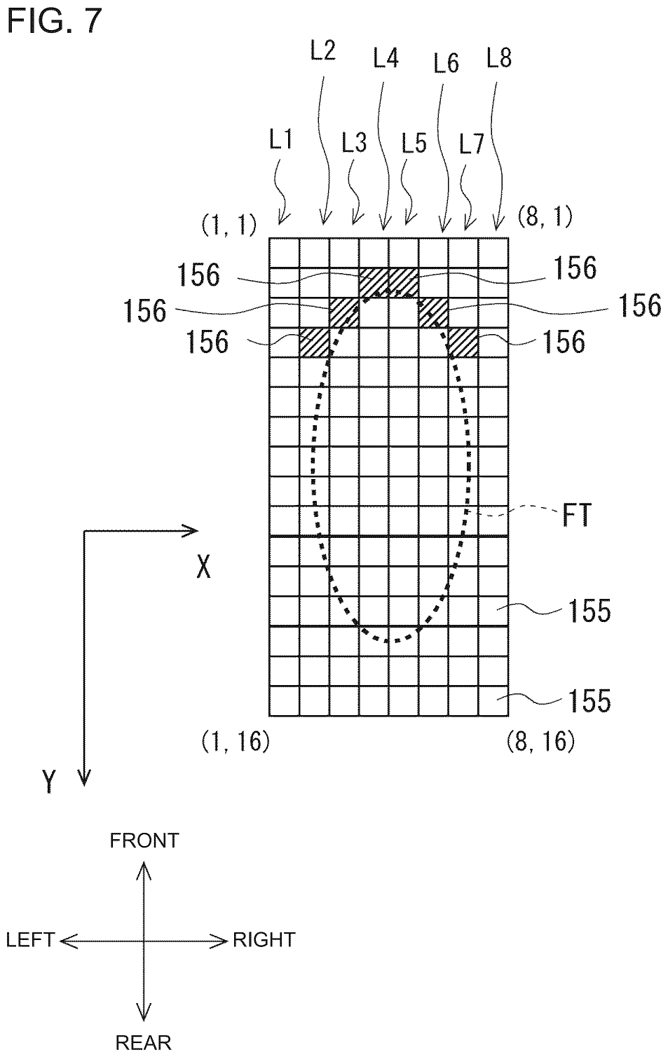

The correction process of the correction unit 172 will be described in detail using FIG. 7. FIG. 7 is a top view schematically showing the arrangement of the cells of the load distribution sensor 152. Of the sensors 155 of the load distribution sensor 152, FIG. 7 shows only the sensors 155 in the region on which the foot FT has landed and in a part surrounding that region. Specifically, in FIG. 7, eight sensors 155 in the left-right direction×16 sensors 155 in the front-rear direction are shown, and the load from the foot FT is applied to some of these sensors. Thus, the load is not applied to the sensors 155 on the outside of the region on which the foot FT has landed, and therefore the sensor outputs thereof are 0.

In FIG. 7, the row on the leftmost side is referred to as a line L1. The line L1 has a plurality of sensors (cells) 155 arranged along the front-rear direction. Similarly, the second row to the eighth row from the left side are referred to as a line L2 to a line L8, respectively. The load distribution sensor 152 sequentially reads data from the row on the left side. For example, the load distribution sensor 152 reads the data of the line L2 after finishing reading the data of the line L1. The load distribution sensor 152 reads the data in the order of L1, L2, L3, L4, L5, L6, L7, and L8.

The cell addresses of the 8×16 sensors 155 of the load distribution sensor 152 are represented by XY-addresses. Specifically, the X-address represents a position in the left-right direction, and the Y-address represents a position in the front-rear direction. The X-addresses of the sensors 155 on the leftmost side are 1, and the X-addresses of the sensors on the rightmost side are 8. The Y-addresses of the sensors 155 on the frontmost side are 1, and the Y-addresses of the sensors on the rearmost side are 16. Thus, the XY-address of the sensor 155 at the upper left corner is (1, 1). The XY-address of the sensor 155 at the upper right corner is (8, 1). The XY-address of the sensor 155 at the lower left corner is (1, 16). The XY-address of the sensor 155 at the lower right corner is (8, 16). The line L1 includes the sensors 155 of (1, 1) to (1, 16).

A range in which the foot FT has landed is from the line L2 to the line L7. The sensors 155 in the line L1 and the sensors 155 in the line L8 do not detect the load. Here, of the cells (sensors 155) in the lines L2 to L7 that detect the load, the cells located on the frontmost side are referred to as leading cells 156.

For example, the leading cell 156 in the line L2 is the fourth sensor 155 from the front side, and the leading cell 156 in the line L3 is the third sensor 155 from the front side. Thus, the XY-address of the leading cell 156 in the line L2 is (2, 4), and the XY-address of the leading cell 156 in the line L3 is (3, 3). Similarly, the addresses of the leading cells 156 in the lines L4 to L7 are (4, 2), (5, 2), (6, 3), and (7, 4), respectively. The leading cell 156 serves as a reference in correction. The Y-address of the leading cell 156 is also referred to as a leading address. Accordingly, L increases toward the rear side, with the leading cell 156 being 1.

The correction unit 172 calculates the corrected value C by applying Formula (1) on a line-by-line basis. The correction unit 172 corrects the sensor outputs according to the sensor address in the front-rear direction. Specifically, the correction unit 172 determines the sensor at the front end of the region of landing on a line-by-line basis. Then, the correction unit 172 uses, as a reference, the sensor 155 at the front end of each line as the leading cell 156.

For example, as shown in FIG. 7, the Y-address of the leading cell 156 differs among the lines. The leading address differs according to the line. The correction unit 172 determines the leading address of each line. For example, the correction unit 172 can use in one line, as the leading address, the Y-address of the cell that is located on the frontmost side among cells of which the sensor outputs are equal to or larger than a threshold value. The correction unit 172 corrects the sensor outputs line by line with reference to the leading address of each line. The correction unit 172 corrects the sensor outputs of the sensors 155 in one line using the correction factor α according to the sensor address in the front-rear direction with reference to the leading address.

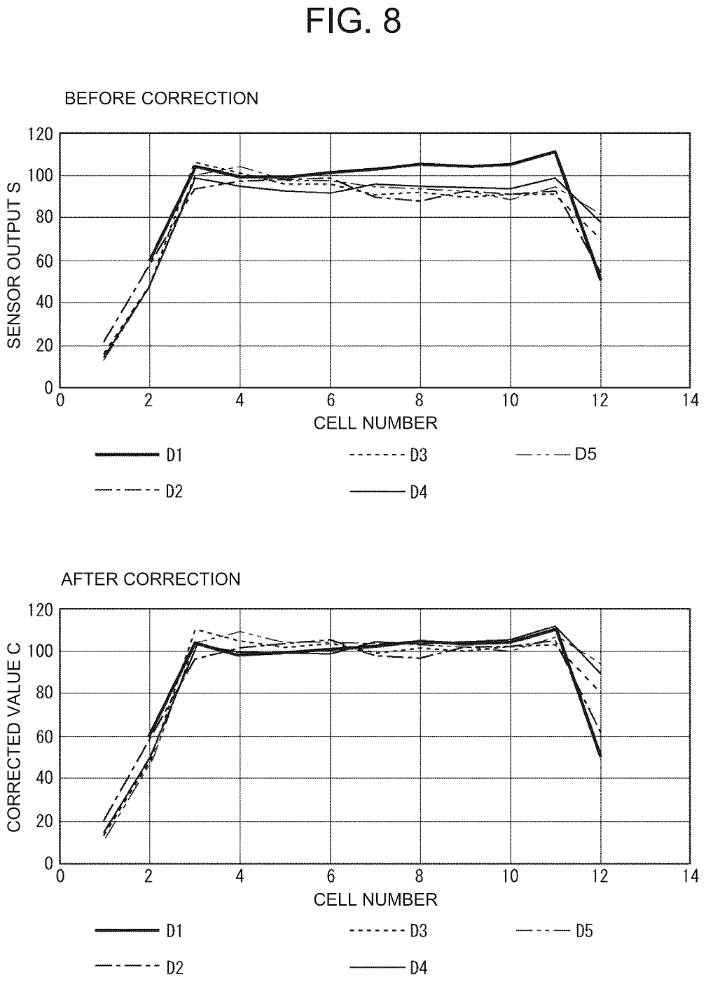

As shown in Formula (2), the correction factor α becomes larger farther away rearward from the leading cell 156 serving as a reference. Therefore, correction can be appropriately performed. As has been described using FIG. 5, the sensor output tends to be larger at the rear end of the foot FT than at the front end. Therefore, correction can be appropriately performed. FIG. 8 is graphs showing sensor outputs S before correction and sensor outputs after correction (corrected values C). The axis of abscissas represents the cell number L, and the axis of ordinates represents the sensor output S or the corrected value C. In FIG. 8, measurement results in the case where a uniform load was applied to the load distribution sensor 152 are shown.

In FIG. 8, data D1 represents data of when the treadmill 131 is not operating. That is, the data D1 represents data of when the moving speed of the belt 132 is 0 km per hour. Data D2 to D5 represent data of when the moving speed of the belt 132 is 2 km per hour. The cell address of the leading cell is a cell number “1.”

In the data D2 to D5 before correction, the sensor output S is lower than in data D1. In particular, in the data D2 to D5 before correction, the sensor output S becomes smaller as the cell number becomes larger. For this reason, as described above, the correction factor α is set so as to become larger as the cell number L becomes larger. Thus, the corrected values C become uniform regardless of the cell number. The correction unit 172 can appropriately correct the cell outputs.

Description of FIG. 6 will be resumed. The value-of-load calculation unit 174 calculates the value of the load received from the foot based on the corrected value C of each cell. Since the corrected value C is obtained for each cell, it is two-dimensional data. The value-of-load calculation unit 174 can obtain the value of the load by integrating the corrected values C that are two-dimensional data. The value-of-load calculation unit 174 calculates the value of the load based on the sum of the corrected values that are two-dimensional data. The value-of-load calculation unit 174 calculates the value of the load representing the load received from the foot FT and outputs that value to the determination unit 175.

The determination unit 175 performs determination relating to the phase in the walking cycle based on the estimated value of the load. The determination unit 175 compares the value of the load with a threshold value that is set beforehand. The overall control unit 210 determines a timing at which the value of the load falls below the threshold value as a switching timing at which the contact leg transitions to the idling leg. The determination unit 175 performs the determination to detect a timing of shift of weight from the affected leg.

The driving control unit 176 controls the joint driving unit 221 according to the determination result of the determination unit 175. For example, the driving control unit 176 outputs a control signal to the joint driving unit 221 such that the joint driving unit 221 operates at the switching timing. An actuator of the joint driving unit 221 operates in response to the control signal. The joint driving unit 221 can bend the knee joint at the switching timing, i.e., the timing of shift of weight from the affected leg.

In this way, the joint driving unit 221 can provide an assistance force according to the walking phase. Therefore, the trainee 900 can undergo effective gait training. The control target to be controlled according to the determination result is not limited to the joint driving unit 221. For example, the driving control unit 176 may control the front-side pulling unit 135 and the rear-side pulling unit 137 such that the pulling forces of the front-side wire 134 and the rear-side wire 136 act according to the walking phase. Thus, the front-side pulling unit 135 and the rear-side pulling unit 137 can provide assistance for swinging forward at the timing of shift of weight.

Even in the state where the treadmill is operating, the value of the load can be estimated with high accuracy. That is, even when the load distribution sensor 152 that detects a load applied through the belt 132 is used, the walking motion can be appropriately analyzed. The driving control unit 176 can perform the driving control at an appropriate timing. The trainee 900 can undergo effective training.

Further, the value-of-load calculation unit 174 may have a CNN that performs a convolution process using two-dimensional data including the corrected values C of the respective cells as an input. Then, the value of the load can be estimated with higher accuracy. This will be described.

As shown in FIG. 5 etc., the viscoelastic sheet 1512 deforms also at a boundary part between a region where the foot FT is present and a region where the foot FT is not present. That is, the viscoelastic sheet deforms at a site which is not directly under the foot FT and at which the load is not applied from the foot FT (e.g., a site on the front side of the position P1 where the conductive sheet 1513 slants). At the boundary part, therefore, the sensors 155 that are not directly under the foot FT detect the load (hereinafter also referred to as an involvement phenomenon).

Thus, the sensors 155 detect the pressure over a region larger than the actual size of the foot FT as grounded. The pressure (load) is detected as the viscoelastic sheet 1512 deforms at a larger number of cells than the number of cells that are actually directly under the foot FT. In other words, it is not possible to distinguish between cells that are crushed and cells that are not crushed based on the output values of the respective sensors 155 alone.

The value-of-load calculation unit 174 uses a machine learning model having a CNN. The two-dimensional data of the corrected values of the plurality of cells is input into the CNN. Having a two-dimensional kernel as a weight, the CNN can add weight with the corrected values of adjacent cells taken into account.

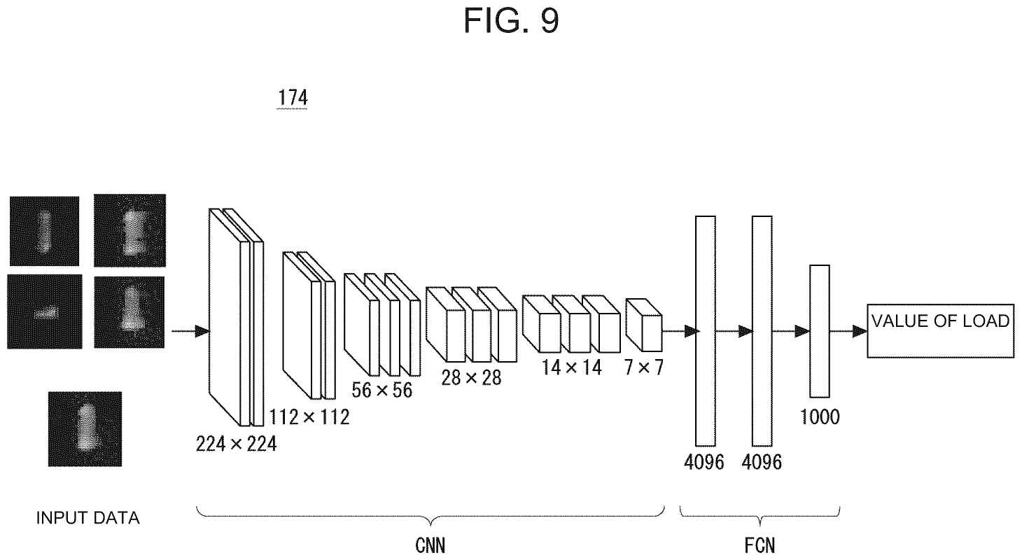

In the following, the learning model used by the value-of-load calculation unit 174 will be described. FIG. 9 is a conceptual view for describing one example of the learning model. The learning model is an inference model generated by machine learning.

The learning model is a deep learning (DL) model having a plurality of layers. As described above, the input data for the learning model is two-dimensional data. Therefore, the input data is represented by two-dimensional grayscale images.

The learning model includes convolutional neural networks (CNNs) that preform a convolution process on the two-dimensional data. The CNNs include a convolution layer, a pooling layer, etc. At a stage after the CNNs, fully connected networks (FCNs) including a fully connected layer is provided. Of course, another network may be used for the learning model.

The CNN performs a convolution process on the two-dimensional data including the corrected values of the respective cells. Having a two-dimensional kernel as a weight, the convolutional neural network CNN can perform a weighting process with adjacent cells taken into account. In the convolutional neural network, a feature quantity according to the two-dimensional data is extracted. The estimation accuracy of the value of the load can be thereby improved. The weight of the CNN etc. can be calculated by machine learning.

As has been described, the load distribution sensor 152 has the plurality of sensors 155 arranged in a two-dimensional array and detects the distribution of the load applied from the trainee 900. The value-of-load calculation unit 174 uses the two-dimensional data based on the corrected values as an input. The value-of-load calculation unit 174 has the CNN that performs a convolutional calculation process, and estimates the value of the load received from the user. The output values of the respective sensors 155 are multi-bit data, and therefore the input data for the value-of-load calculation unit 174 is two-dimensional data similar to image data. In other words, the sensor output is similar to image data, with the corrected value of one sensor 155 constituting pixel data of one pixel. The CNN can perform a convolution process in the same manner as a process of image recognition etc. Since a CNN for image processing can be used, versatility can be improved.

Thus, the value-of-load calculation unit 174 can obtain the value of the load with high accuracy. There is no need for the value-of-load calculation unit 174 to perform processing by distinguishing between a region that is actually crushed and a region that is deformed due to the involvement phenomenon. As a result, the walking phase can be appropriately analyzed, and effective training becomes possible.

In this embodiment, the load distribution sensor 152 is disposed on the lower side of the belt 132. The sensors 155 detect the load (pressure) applied through the belt 132. Also in such a case, the value-of-load calculation unit 174 can estimate the value of the load with good accuracy. This is because parameters such as the weight of the CNN have been learned based on the assumption of the case where the belt 132 moves. That is, the parameter of weighting has been optimized in the state where the belt 132 is moving. Thus, the estimation accuracy of the value of the load can be improved. By using the machine learning model having the CNN, the value-of-load calculation unit 174 can calculate the value of the load with higher estimation accuracy. As a result, the overall control unit 210 can appropriately analyze the walking phase, and the trainee 900 can undergo effective training.

Further, the driving control unit 176 controls the leg brace 120 that is a robot leg based on the value of the load estimated by the value-of-load calculation unit 174. For example, the driving control unit 176 outputs a control signal for controlling the joint driving unit 221 for the knee joint etc. The driving control unit 176 can drive the leg brace 120 according to the walking phase. Since the leg brace 120 can assist the walking motion with an appropriate assistance force, the trainee 900 can undergo effective training.

The degree of the involvement phenomenon can vary according to the moving speed of the belt 132. Therefore, the moving speed of the belt 132 may be used as an input for the value-of-load calculation unit 174. Thus, the two-dimensional data and the moving speed are input data for the learning model. For example, the learning model may use a different parameter according to the moving speed of the belt 132. Specifically, an activating function, a filter, a weight, etc. may be switched. Or the value-of-load calculation unit 174 can use a learning model for high speed and a learning model for low speed by switching between them. When generating a learning model by machine learning, a learning device should perform machine learning for each moving speed of the belt 132. In other words, the learning device uses, as learning data, the data on the moving speed in association with the two-dimensional data of the sensor outputs.

The sensor output acquisition unit 171 may trim the sensor outputs from all the sensors 155 included in the load distribution sensor 152. The sensor output acquisition unit 171 may then input the trimmed two-dimensional data into the value-of-load calculation unit 174. For example, a trimming size may be set beforehand in the sensor output acquisition unit 171. Here, the trimming size can be (k×1) rectangular regions. k is larger than 1 and smaller than m, and l is larger than 1 and smaller than n. The sensor output acquisition unit 171 cuts out k×1 pieces of two-dimensional data centered at the center of gravity of the distribution of the load in the load distribution sensor 152. Thus, the sensor outputs are (k×1) pieces of two-dimensional map data. In this way, the amount of data processing can be reduced, so that the processing speed can be increased. The value-of-load calculation unit 174 can calculate the value of the load on a frame-by-frame basis.

The sensor output acquisition unit 171 or the correction unit 172 may function as a pressure conversion unit that converts the sensor outputs or the corrected values into pressure values. In this case, the value-of-load calculation unit 174 uses two-dimensional map data composed of pressure values as input data. Specifically, the load distribution sensor 152 performs A/D conversion of the output values (voltage values) output from the respective sensors 155 into digital data. Then, the sensor output acquisition unit 171 converts the sensor outputs that are digital data into pressure values using a conversion formula or a conversion table. The load distribution sensor 152 has a conversion formula or a conversion table for converting measured values of electrical characteristics into pressure values.

The conversion table or the conversion formula may show a conversion result of a linear shape, or may show a conversion result of a shape other than a linear shape. For example, the conversion formula may be a second-degree or higher-degree polynomial formula. The conversion formula or the conversion table for converting the sensor output values into pressure values may be different for each sensor 155, or may be the same for the plurality of sensors 155. For example, the sensor output acquisition unit 171 can use a conversion formula that has been calibrated for each sensor 155. The estimation accuracy can be thereby improved.

The overall control unit 210 is not limited to a physically single device. That is, the sensor output acquisition unit 171, the value-of-load calculation unit 174, the determination unit 175, the gain setting unit 178, and the driving control unit 176 may be dispersedly disposed in a plurality of devices. In this case, data should be transmitted and received among the devices through a network. For example, the gain setting unit 178 may be installed in a server or the like so as to be available to a plurality of gait training devices 100.

The above-described control method can be realized in part or in whole by a computer program. The program can be supplied to a computer by being stored using various types of non-transitory computer-readable media. Non-transitory computer-readable media include various types of tangible storage media. Examples of non-transitory computer-readable media include magnetic recording media, magnetooptical recording media, CD-ROMs (read-only memories), CD-Rs, CD-R/Ws, and semiconductor memories. Examples of magnetic recording media include flexible disks, magnetic tapes, and hard disk drives. Examples of magnetooptical recording media include magnetooptical disks. Examples of semiconductor memories include mask ROMs, programmable ROMs (PROMs), erasable PROMs (EPROMs), flash ROMs, and random-access memories (RAMs). Further, the program may be supplied to a computer by various types of transitory computer-readable media. Examples of transitory computer-readable media include electrical signals, optical signals, and electromagnetic waves. Transitory computer-readable media can supply the program to a computer through a wired communication path, such as an electrical wire or an optical fiber, or through a wireless communication path.

What is claimed is:

1. A gait training system comprising:
a treadmill having a belt on which a user lands;
a load distribution sensor that has a plurality of sensors arranged in a two-dimensional array and detects a load applied through the belt;
a sensor output acquisition unit that acquires sensor outputs from the sensors of the load distribution sensor;
a correction unit that corrects the sensor outputs from the respective sensors based on a moving speed of the belt; and
a value-of-load calculation unit that calculates a value of a load received from the user based on corrected values corrected by the correction unit.

2. The gait training system according to claim 1, wherein the correction unit determines a sensor at a front end of a region on which the user has landed on a line-by-line basis, and corrects the sensor output according to a sensor address in a front-rear direction with reference to a sensor address of the sensor at the front end.

3. The gait training system according to claim 1, wherein the correction unit corrects the sensor outputs of the sensors in one row along the front-rear direction using a correction factor according to the sensor address in the front-rear direction.

4. The gait training system according to claim 1, wherein the load distribution sensor sequentially reads outputs of the sensors in one row arranged in the front-rear direction.

5. The gait training system according to claim 1, further comprising:
a robot leg worn on a leg of the user; and
a driving control unit that drives the robot leg based on the value of the load.

6. The gait training system according to claim 1, wherein the value-of-load calculation unit has a CNN that performs a convolution process using two-dimensional data including the corrected values of the plurality of cells as an input.

7. A control method of a gait training system including:
a treadmill having a belt on which a user lands; and
a load distribution sensor that has a plurality of sensors arranged in a two-dimensional array and detects a load applied through the belt,
the control method comprising the steps of:
acquiring sensor outputs from the sensors of the load distribution sensor;
correcting the sensor outputs from the respective sensors based on a moving speed of the belt to obtain corrected values; and
calculating a value of a load received from the user based on the corrected values.

8. The control method of a gait training system according to claim 7, wherein, in the step of correcting, a sensor at a front end of a region on which the user has landed is determined on a line-by-line basis, and the sensor output is corrected according to a sensor address in a front-rear direction with reference to a sensor address of the sensor at the front end.

9. The control method of a gait training system according to claim 7, wherein the sensor outputs of the sensors in one row along the front-rear direction are corrected using a correction factor according to the sensor address in the front-rear direction.

10. The control method of a gait training system according to claim 7, wherein the load distribution sensor sequentially reads outputs of the sensors in one row arranged in the front-rear direction.

11. The control method of a gait training system according to claim 7, wherein the gait training system further includes:
a robot leg worn on a leg of the user; and
a driving control unit that drives the robot leg based on the value of the load.

12. The control method of a gait training system according to claim 7, wherein, in the step of calculating the value of the load, a CNN performs a convolution process using two-dimensional data including the corrected values of the plurality of cells as an input.

13. A non-transitory storage medium storing a control program that makes a computer execute a control method for controlling a gait training system including:
a treadmill having a belt on which a user lands; and
a load distribution sensor that has a plurality of sensors arranged in a two-dimensional array and detects a load applied through the belt,
the control method comprising the steps of:
acquiring sensor outputs from sensors of the load distribution sensor;
correcting the sensor outputs from the respective sensors based on a moving speed of the belt to obtain corrected values; and
calculating a value of a load received from the user based on the corrected values.
